(19) United States
(12) Patent Application Publication
(10) Pub. No.: US 2008/0015386 A1
(43) Pub. Date: Jan. 17, 2008

(54) Crystal of L-Ornithine-Citric Acid Salt

(75) Inventors: Hideki Murata, Hofu-shi (JP); Yu Yamamoto, Fukuoka (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., CHIYODA-KU TOKYO JAPAN (JP)

(21) Appl. No.: 11/571,072

(22) PCT Filed: Jun. 24, 2005
(86) PCT No.: PCT/JP05/11635

§ 371 Date: Dec. 21, 2006

(30) Foreign Application Priority Data
Jun. 25, 2004 (JP) 2004-188813

(51) Int. Cl. C07C 229/26

(52) U.S. Cl. 562/561

(57) ABSTRACT

The present invention provides a crystal of a salt of L-ornithine and citric acid, the crystal of the salt of L-ornithine and citric acid in which pH of the 5 weight % aqueous solution is 3 to 6, the crystal of the salt of L-ornithine and citric acid in which a composition ratio of L-ornithine and citric acid is 2:1 (molar ratio), a process for producing the crystals of the salt of L-ornithine and citric acid which comprises dissolving L-ornithine and citric acid in water, and precipitating the crystal from the resulting aqueous solution, the process for producing the crystal of the salt of L-ornithine and citric acid in which the pH of the aqueous solution is 3 to 6, and the like.

### TECHNICAL FIELD

The present invention relates to a crystal of a salt of L-ornithine and citric acid (L-ornithine-citric acid salt) and a process for producing the same.

### BACKGROUND ART

L-ornithine has been widely used as an ingredient of nutrition enriching additives, pharmaceuticals or the like.

Since it is difficult to prepare a free base of L-ornithine as a crystal, it is available usually in the form of a salt such as hydrochloride (Product Catalogue 2004 to 2005 of Sigma).

When L-ornithine is used as an ingredient of a transfusion or the like for the purpose of nutrition enriching or the like, for example, by using its hydrochloride is used as it is, an acidosis symptom may be induced. Also, administration of a transfusion containing a large amount of chlorine ions is unfavorable for patients with a renal disease in particular. It has also been well known that when L-ornithine is used either by being mixed in foods or the like as a nutrition enriching additive or the like or orally as it is, it is difficult to utilize the same in the form of, for example, hydrochloride because of its bitter taste.

As a salt of L-ornithine, for example, α-ketoglutarate (refer to Patent Document 1), L-aspartate (refer to Patent Document 2), malate (refer to Patent Document 3) and the like other than the hydrochloride described above are known as crystals.

Meanwhile, it is known that a salt of ornithine and citric acid can be used as a taste improver, but crystals thereof are not known yet (refer to Patent Document 4).

Patent Document 1: gazette of Japanese Published Examined Patent Application No. 3194/1971

Patent Document 2: gazette of Japanese Published Unexamined Patent Application No. 364155/1992

Patent Document 3: gazette of Japanese Published Unexamined Patent Application No. 136254/1980

Patent Document 4: gazette of Japanese Published Unexamined Patent Application No. 144088/2003

### DISCLOSURE OF THE INVENTION

Problems to be Solved by the Invention

An object of the present invention is to provide a crystal of an L-ornithine-citric acid salt excellent as a supply source of L-ornithine and a process for producing the same.

Means for Solving the Problems

The invention relates to the following (1) to (10).

(1) A crystal of a salt of L-ornithine and citric acid.

(2) The crystal according to (1), wherein pH of a 5 weight % aqueous solution of the crystals is 3 to 6.

(3) The crystal according to (1), wherein a composition ratio of L-ornithine and citric acid is 2:1 (molar ratio).

(4) A process for producing the crystal according to any one of (1) to (3), which comprises dissolving L-ornithine and citric acid in water, and precipitating the crystal from the resulting aqueous solution.

(5) The process according to (4), wherein the pH of the aqueous solution is 3 to 6.

(6) The process according to (4), wherein the pH of the aqueous solution is 4 to 5.

(7) The process according to any one of (4) to (6), wherein the method of precipitating the crystal is a method comprising a step of adding a hydrophilic organic solvent to the aqueous solution.

(8) The process according to (7), wherein the hydrophilic organic solvent is an alchols, an amides, acetone or acetonitrile.

(9) The process according to (7), wherein the hydrophilic organic solvent is a solvent selected from the group consisting of methanol, ethanol, propanol, isopropyl alcohol, butanol, ethylene glycol, diethylene glycol, N,N-dimethylformamide, N,N-dimethylacetamide, N-methylpyrrolidone, acetone and acetonitrile.

(10) The process according to (7), wherein the hydrophilic organic solvent is methanol or ethanol.

Effect of the Invention

The present invention provides a crystal of an L-ornithine-citric acid salt excellent as a supply source of L-ornithine and a process for producing the same.

### BRIEF DESCRIPTION OF THE DRAWING

FIG. 1 shows test results of hygroscopicity of the crystals of an L-ornithine-½ citric acid salt obtained in Example 1. In the drawing, the abscissa represents the number of days elapsed (day) after starting the test, and the ordinate represents a hygroscopic rate (%).

### BEST MODE FOR CARRYING OUT THE INVENTION

The crystal of the L-ornithine-citric acid salt in the present invention comprises preferably 0.5 to 3 mols of L-ornithine and 1 mol of citric acid, more preferably 2 mols of L-ornithine and 1 mol of citric acid. The aqueous solution thereof is neutral or acidic solution. In the 5 weight % aqueous solution thereof, the pH of the solution is 3 to 7, preferably 3 to 6, more preferably 4 to 5, most preferably 4.6 to 5.0.

While the crystal usually exists singly, it may exist as a solvate with water or various organic solvents, and these solvates are also comprised in the present invention.

Examples of the hydrophilic organic solvent used in the invention include alchols such as methanol, ethanol, propanol, isopropyl alcohol, butanol, ethylene glycol or diethylene glycol; amides such as N,N-dimethylformamide, N,N-dimethylacetamide or N-methylpyrrolidone; acetonitrile; acetone or the like. Methanol or ethanol is preferable. These solvents can be used alone or in combination thereof.

The process for producing the crystals of the L-ornithine-citric acid salt in the present invention is described below.

For example, a culture solution, a concentrated culture solution or the like containing a salt of L-ornithine such as L-ornithine hydrochloride or L-ornithine which is commercially available is treated with a strongly acidic ion exchange resin and the like to obtain an aqueous solution containing a free base of L-ornithine. Citric acid is added to the resulting aqueous solution of a free base of L-ornithine, and dissolved therein. To obtain the crystal of the present invention in which an L-ornithine to citric acid composition ratio is, for example, 2:1 (molar ratio), citric acid is added in an amount of 0.3 to 1 equivalent, preferably 0.4 to 0.6 equivalent, most preferably 0.5 equivalent to L-ornithine, and the resulting solution is adjusted to pH of, for example, 3 to 7, preferably 3 to 6, more preferably 4 to 5, most preferably 4.6 to 5.0.

In order to efficiently obtain the crystal of the L-ornithine-citric acid salt from the resulting aqueous solution, preferably the crystals are isolated by precipitation thereof according to cooling the aqueous solution to −10 to 20° C., addition of a seed crystal to the aqueous solution, addition of the hydrophilic organic solvent to the aqueous solution, or addition of the aqueous solution to the hydrophilic organic solvent. Alternatively, the crystals may be precipitated by conducting a combination of these methods. In case of addition of the hydrophilic organic solvent to the aqueous solution or addition of the aqueous solution to the hydrophilic organic solvent, the crystals may partially be precipitated in the aqueous solution.

In case of addition of the hydrophilic organic solvent to the aqueous solution to precipitate the crystals, the aqueous solution is preferably used by conducting a procedure such as evaporation for adjusting the concentration of the L-ornithine-citric acid salt to 30 to 80%, preferably 40 to 60%. When the aqueous solution having the concentration of 30% or more is used, the amount of the hydrophilic organic solvent used to precipitate the crystals can be decreased, which is more preferably. When the aqueous solution having the concentration of 80% or less is used, the aqueous solution and the hydrophilic organic solvent are mixed more easily, and the precipitation of the crystals is induced easily. In addition, conversion of the precipitated crystals to a blocked aggregate is easily avoidable. The hydrophilic organic solvent is used in an amount which is usually 1 to 8 times (water content is approximately 50% to 11%), preferably 1.5 to 5 times (water content is approximately 40 to 17%), more preferably 2 to 3 times (water content is approximately 33 to 25%) as large as the amount of the above aqueous solution which is adjusted the concentration. And the solvent is added gradually or dropwise at −10° C. to 60° C., preferably −10° C. to room temperature to the above aqueous solution which is adjusted the concentration. The crystals are sometimes precipitated by mere addition of the hydrophilic organic solvent, but, the crystals are usually precipitated by stirring the resulting mixed solution at −10° C. to room temperature for 5 minutes to 72 hours. The crystals separately obtained by the present invention can be used as seed crystals and added to the resulting mixed solution to induce the precipitation of the crystals. Seed crystals are sufficient so long as crystallization can be induced, and used in an amount of 0.01 to 0.1%, preferably 0.05% to the amount of the L-ornithine-citric acid salt contained in the mixed solution.

In case of addition of the aqueous solution to the hydrophilic organic solvent to precipitate the crystals, the crystals can be precipitated by dropping the aqueous solution which is adjusted the concentration to the hydrophilic organic solvent at −10° C. to 60° C., preferably −10° C. to room temperature with stirring described above. The hydrophilic organic solvent is usually used in an amount which is 1 to 8 times as large as the amount of the above aqueous solution which is adjusted the concentration.

The crystals of the L-ornithine-citric acid salt can be obtained by separating the precipitated crystals via filtration or the like and drying the same.

Crystals of a salt of another basic amino acid (for example, lysine, arginine and the like) and citric acid may be obtained in the same manner.

Next, the hygroscopicity of the crystals of the L-ornithine-citric acid salt in the present invention is described below in Test Example.

### TEST EXAMPLE 1

A saturated aqueous solution of sodium chloride (approximately 200 mL) was allowed to stand in a plastic desiccator at 25° C. for 24 hours (relative humidity was adjusted to 75%). The crystals (2 g) prepared in Example 1 were weighed out in a glass weighing vessel. The weighing vessel was allowed to stand in the desiccator with the humidity adjusted, and the weight change of the weighing vessel was measured over the course of time.

The hygroscopicity (hygroscopic degree) was calculated using the following formula (1) on the basis of the weight change at each measuring time. The measurement of the weight change of the weighing vessel was continued until day 7.

[Formula 1]
Hygroscopic rate (%)=W3−W2/W2−W1×100 (1)

W1=Weight (g) of a weighing vessel

W2=Weight (g) of a weighing vessel filled with crystals before the test

W3=Weight (g) of a weighing vessel filled with crystals after the test

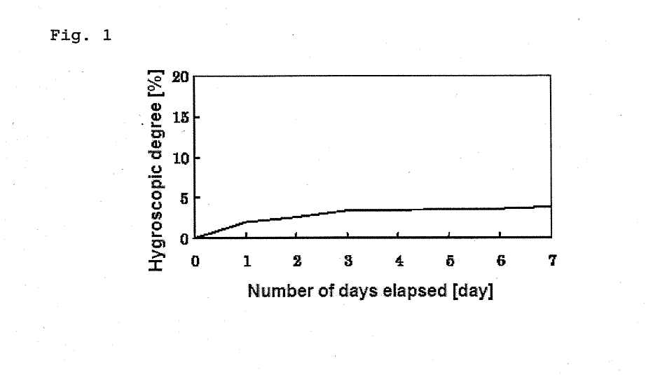

The results are shown in FIG. 1.

The crystals of the L-ornithine-citric acid salt prepared in Example 1 did not show notable hygroscopicity. Therefore, it was shown that there was no need to pay special attention to humidity in storage of the L-ornithine-citric acid salt of the present invention.

Also, the L-ornithine-citric acid salt prepared in Example 3 did not have a bitter taste.

As described above, the crystals of the L-ornithine-citric acid salt in the present invention can be stored in an ambient atmosphere at room temperature. The crystals of the L-ornithine-citric acid salt are excellent as a supply source of L-ornithine because during the process for producing the same, corrosive chemicals such as hydrochloric acid are not used and citric acid is less costly in comparison with L-malic acid and the like.

Moreover, the crystals of the L-ornithine-citric acid salt are expected to have properties provided by citric acid in addition to the effect inherent in L-ornithine. Citric acid is an intermediate of a citric acid cycle (TCA cycle). Through activation thereof, an effect of recovery from fatigue can be expected by acceleration of fatty acid synthesis and elimination of lactic acid accumulated in the body. Further, improvement of a bitter taste provided by hydrochloride or the like is also expected. Therefore, the crystals of the L-ornithine-citric acid salt are expected to be used as a better supply source of L-ornithine in nutrition enriching additives or pharmaceuticals.

The present invention is illustrated specifically below by referring to Examples. However, the invention is not limited to these Examples.

### EXAMPLE 1

L-ornithine hydrochloride (20 g, 15.7 g as free L-ornithine) was dissolved in water (400 mL), and the solution was passed through a column filled with a strongly acidic ion exchange resin (Marason C, H-type, 200 mL). After the resin was washed with water (200 mL), L-ornithine was eluted with 2 mol/L aqueous ammonia (400 mL). The eluate was concentrated to approximately 200 mL under reduced pressure, and citric acid (12 g, 0.5 equivalent to L-ornithine) was then added to the resulting aqueous solution of L-ornithine (containing 0.119 mol of L-ornithine) to adjust the pH of the aqueous solution to 4.7. The resulting aqueous solution was concentrated under reduced pressure to adjust the total volume to 45 mL. Subsequently, ethanol (105 mL) was gradually added to the solution with stirring at room temperature. After the mixture was further stirred at room temperature for 20 hours, the precipitated crystals were collected by filtration, and washed with ethanol. The resulting crystals were dried overnight at 20° C. under reduced pressure to give crystals of L-ornithine-½ citric acid salt (25.1 g, yield: 92.7%) as pale yellow massive crystals.

Melting point: 165.5° C.

IR spectrum (KBr, cn⁻¹): 1288.4, 1033.8, 948.9, 803.3, 715.5.

X-ray powder diffraction analysis: it was measured by RAD-X type (manufactured by Rigaku Denki). The results are shown in Table 1.

[Table 1]

TABLE 1

| Analytical angle | Peak intensity |
|---|---|
| (2θ (theta)) | (Relative intensity) |
| 6.05 | 21 |
| 11.50 | 62 |
| 12.15 | 32 |
| 18.65 | 78 |
| 20.70 | 100 |
| 23.45 | 44 |
| 24.55 | 39 |
| 25.30 | 45 |
| 29.65 | 34 |
| 30.35 | 34 |

Analysis of crystal composition: the results are shown in Table 2.

[Table 2]

TABLE 2

| | Found (%)* | Calculated (%)** |
|---|---|---|
| L-ornithine(%) | 56.5 | 57.9 |
| Citric acid(%) | 43.5 | 42.1 |

(Notes)
*Each found value was calculated by analyzing L-ornithine via an OPA coloring method (excitation wavelength: 340 nm, fluorescence wavelength: 455 nm) and citric acid via a UV detection method (detection wavelength: 210 nm) using high-performance liquid chromatography (HPLC).
**Each calculated value was calculated as (C₅H₁₂N₂O₂)₂•C₆H₈O₇.

### EXAMPLE 2

To an aqueous solution of L-ornithine (containing 0.119 mol of L-ornithine) prepared in the same manner as in Example 1 was added citric acid (12 g, 0.5 equivalent to L-ornithine) to adjust pH of the aqueous solution to 4.7. The resulting solution was concentrated under reduced pressure to adjust the total volume to 45 mL. Subsequently, methanol (105 mL) was gradually added to the solution with stirring at room temperature. After the mixture was further stirred at room temperature for 18 hours, the precipitated crystals were collected by filtration, and washed with methanol. The resulting crystals were dried overnight at 20° C. under reduced pressure to give crystals of an L-ornithine-½ citric acid salt (24.8 g, yield: 91.6%) as pale yellow flaky crystals.

Melting point: 161.8° C.

IR spectrum (KBr, cn⁻¹): 1287.4, 1044.4, 935.4, 808.1, 754.1.

X-ray powder diffraction: it was measured by RAD-X type (manufactured by Rigaku Denki). The results are shown in Table 3.

[Table 3]

TABLE 3

| Analytical angle | Peak intensity |
|---|---|
| (2θ (theta)) | (Relative intensity) |
| 6.50 | 33 |
| 7.00 | 21 |
| 13.05 | 22 |
| 14.05 | 100 |
| 15.50 | 25 |
| 18.50 | 46 |
| 19.35 | 94 |
| 20.70 | 33 |
| 23.75 | 28 |
| 24.60 | 59 |
| 28.40 | 39 |

Analysis of crystal composition: the results are shown in Table 4.

[Table 4]

TABLE 4

| | Found (%)* | Calculated (%)** |
|---|---|---|
| L-ornithine(%) | 57.0 | 57.9 |
| Citric acid(%) | 43.0 | 42.1 |

(Notes)
*Each found value was calculated by analyzing L-ornithine via an OPA coloring method (excitation wavelength: 340 nm, fluorescence wavelength: 455 nm) and citric acid via a UV detection method (detection wavelength: 210 nm) using high-performance liquid chromatography (HPLC).
**Each calculated value was calculated as (C₅H₁₂N₂O₂)₂•C₆H₈O₇.

### EXAMPLE 3

To an aqueous solution of L-ornithine (containing 0.119 mol of L-ornithine) obtained in the same manner as in Example 1 was added citric acid (12 g, 0.5 equivalent to L-ornithine) to adjust pH of the aqueous solution to 4.7. The resulting solution was concentrated under reduced pressure to adjust the total volume to 68 mL. Subsequently, ethanol (272 mL) was gradually added to the solution with stirring at room temperature. After the mixture was further stirred at room temperature for 24 hours, the precipitated crystals were collected by filtration, and washed with ethanol. The resulting crystals were dried overnight at 20° C. under reduced pressure to give crystals of L-ornithine-½ citric acid salt (25.5 g, yield: 94.0%) as pale yellow flaky crystals.

Melting point: 165.9° C.

Analysis of crystal composition: the results are shown in Table 5.

[Table 5]

TABLE 5

| | Found (%)* | Calculated (%)** |
|---|---|---|
| L-ornithine(%) | 57.2 | 57.9 |
| Citric acid(%) | 42.8 | 42.1 |

(Notes)
*Each found value was calculated by analyzing L-ornithine via an OPA coloring method (excitation wavelength: 340 nm, fluorescence wavelength: 455 nm) and citric acid via a UV detection method (detection wavelength: 210 nm) using high-performance liquid chromatography (HPLC).
**Each calculated value was calculated as (C₅H₁₂N₂O₂)₂•C₆H₈O₇.

### INDUSTRIAL APPLICABILITY

The crystal of the L-ornithine-citric acid salt, the process for producing the same and the like provided by the present invention are useful as a supply source of L-ornithine.

1. A crystal of a salt of L-ornithine and citric acid.

2. The crystal according to claim 1, wherein pH of a 5 weight % aqueous solution of the crystals is 3 to 6.

3. The crystal according to claim 1, wherein a composition ratio of L-ornithine and citric acid is 2:1 (molar ratio).

4. A process for producing the crystal according to any one of claims 1 to 3, which comprises dissolving L-ornithine and citric acid in water, and precipitating the crystal from the resulting aqueous solution.

5. The process according to claim 4, wherein the pH of the aqueous solution is 3 to 6.

6. The process according to claim 7, wherein the pH of the aqueous solution is 4 to 5.

7. The process according to claim 5, wherein the method of precipitating the crystal is a method comprising a step of adding a hydrophilic organic solvent to the aqueous solution.

8. The process according to claim 7, wherein the hydrophilic organic solvent is an alchols, an amides, acetone or acetonitrile.

9. The process according to claim 7, wherein the hydrophilic organic solvent is a solvent selected from the group consisting of methanol, ethanol, propanol, isopropyl alcohol, butanol, ethylene glycol, diethylene glycol, N,N-dimethylformamide, N,N-dimethylacetamide, N-methylpyrrolidone, acetone and acetonitrile.

10. The process according to claim 7, wherein the hydrophilic organic solvent is methanol or ethanol.

11. The process according to claim 8, wherein the pH of the aqueous solution is 4 to 5.

12. The process according to claim 9, wherein the pH of the aqueous solution is 4 to 5.

13. The process according to claim 10, wherein the pH of the aqueous solution is 4 to 5.
